(19) United States
(12) Patent Application Publication
(10) Pub. No.: US 2025/0221833 A1
(43) Pub. Date: Jul. 10, 2025

(54) SILICONE GEL AND ELASTOMER COMBINATION BREAST PROSTHESIS

(71) Applicant: American Breast Care, LP, Marietta, GA (US)

(72) Inventors: Amanda Wollnick, Doraville, GA (US); Robert Halley, Atlanta, GA (US)

(21) Appl. No.: 19/013,613

(22) Filed: Jan. 8, 2025

Related U.S. Application Data
(60) Provisional application No. 63/619,057, filed on Jan. 9, 2024.

(51) Int. Cl. A61F 2/52; A61F 2/50

(52) U.S. Cl. CPC A61F 2/52; A61F 2/5044

(57) ABSTRACT

A breast prosthesis includes a front part that is made of a silicone gel that is encapsulated in a polymer film. A back part that is made from a silicone elastomer is affixed thereto. An alternate form of breast prosthesis includes a front part that is made from a silicone elastomer. A back part that is made of a silicone gel that is encapsulated in a polymer film and that is affixed thereto. In a method of making a breast prosthesis in which a two chamber bag that includes a layer of polytetrafluoroethylene (“PTFE”), a layer of polyurethane and a layer of polyurethane is made. The bag is placed into a mold that defines a breast form-shaped cavity. A silicone gel material is injected into one chamber and a silicone elastomer material is injected into the other chamber. The materials are cured and the PTFE layer is removed.

### CROSS-REFERENCE TO RELATED APPLICATION(S)

This application claims the benefit of U.S. Provisional Patent Application Ser. No. 63/619,057, filed Jan. 9, 2024, the entirety of which is hereby incorporated herein by reference.

### BACKGROUND OF THE INVENTION

### 1. Field of the Invention

The present invention relates to breast prostheses and, more specifically, to a breast prosthesis that includes a silicone gel and an elastomer.

### 2. Description of the Related Art

Many women who have had a mastectomy wear a breast prosthesis at the affected site. Typically, a breast prosthesis is made to look like a natural breast. It is placed against the patient's chest and is typically supported by a brassiere.

Current, commercially available external silicone breast prostheses are either made of silicone gel encapsulated in a polymer film or made of 100% silicone elastomer.

A c soft silicone gel prosthesis includes an envelope made from a thin plastic film into which uncured silicone gel is injected. The pouch and the uncured silicone gel are then placed in a metallic mold having an interior that is complementary in shape to the desired shape of the prosthesis. The mold is then placed in an oven, where the silicone gel is heated until it is cured.

Silicone gel prostheses are soft and mimic the feel and movement of fatty tissue. However, due to the softness and tackiness of the silicone gel, it must be encapsulated in film. Additionally, the prosthesis should be worn in a pocketed mastectomy bra to keep the prosthesis in place against the chest wall. Despite the pocket, there is still possibility for the prosthesis to shift and move around inside the pocket while being worn. This can make the prosthesis uncomfortable to wear. Because the silicone gel prostheses must be worn inside the bra pocket, the pocket fabric can become wet with perspiration while being worn. The prosthesis traps the wet fabric against the chest wall, which can cause skin irritation and make the prosthesis uncomfortable to wear.

Silicone gel prostheses require the gel to be encapsulated in flexible film. In general, silicone elastomers are known to have poor adhesion to most varieties of flexible films. Poor adhesion between the various components of a breast prosthesis will reduce the durability of the prosthesis.

Attachable silicone gel prostheses have a layer of silicone adhesive gel on the back. These attachable prostheses can be worn, directly attached to the chest wall, and supported by a bra. In this embodiment, the bra does not need a pocket. If the wearer does want to wear the attachable prosthesis inside the bra pocket, the adhesive layer will need a cover, or else it will be too sticky to place inside the pocket. However, not all health insurance covers attachable breast prostheses and there is extra maintenance and cleaning to keep the silicone adhesive gel sticky that some breast prosthesis wearers are not inclined to do.

100% silicone elastomer breast prostheses are typically made of a silicone elastomer that has been foamed to make it softer and lighter. The silicone elastomer foam is soft but does not have the same feel or movement as fatty tissue. A 100% silicone elastomer breast prosthesis does not need to be worn inside a pocketed mastectomy bra. The natural tackiness and grippiness of the silicone elastomer can help keep the prosthesis in place while wearing. The wearer can still put the 100% silicone elastomer inside the bra pocket with no cover if desired, unlike an attachable prosthesis. Additionally, there can be potential benefits of having 100% silicone elastomer against the skin, such as scar reduction. Also, there are no wrinkles on the prosthesis front, which is a desired look by some breast prosthesis wearers.

Certain multi-part prostheses can employ both elastomers and gels. However, existing prostheses made from combinations of materials such as elastomers and gels rely on fasteners to hold the various materials together. These prostheses are generally considered to be cumbersome to use.

Therefore, there is a need for a single part breast prosthesis employing both silicone gel and elastomer technologies.

### SUMMARY OF THE INVENTION

In one aspect, the invention is a breast prosthesis that includes a front part that is made of a silicone gel that is encapsulated in a polymer film. A back part that is made from a silicone elastomer is affixed thereto.

In another aspect, the invention is a breast prosthesis that includes a front part that is made from a silicone elastomer. A back part that is made of a silicone gel that is encapsulated in a polymer film and that is affixed thereto.

In another aspect, the invention is a method of making a breast prosthesis in which a two chamber bag that includes a back layer of polytetrafluoroethylene (“PTFE”), a middle layer of polyurethane and a front layer of polyurethane is made. The front layer is heat sealed to the middle layer along a breast form footprint so as to define a front chamber therebetween. The bag is placed into a mold that defines a breast form-shaped cavity and a top is placed against the mold to hold the PTFE layer in place so as to define a back chamber between the middle layer and the PTFE layer. A silicone gel material is injected into the front chamber and a silicone elastomer material is injected into the back chamber. The silicone gel material and the silicone elastomer material are cured so as to form a cured breast prosthesis. The cured breast form is removed from the mold and the PTFE layer is removed from cured breast prosthesis.

In another aspect, the invention is a method of making a breast prosthesis in which a two chamber bag that includes a front layer of polytetrafluoroethylene (“PTFE”), a middle layer of polyurethane and a back layer of polyurethane is made. The back layer is heat sealed to the middle layer along a breast form footprint so as to define a back chamber therebetween. The bag is placed into a mold that defines a breast form-shaped cavity and a top is placed against the mold to hold the PTFE layer in place so as to define a front chamber between the middle layer and the PTFE layer. A silicone gel material is injected into the back chamber and a silicone elastomer material is injected into the front chamber. The silicone gel material and the silicone elastomer material are cured so as to form a cured breast prosthesis. The cured breast form is removed from the mold and the PTFE layer is removed from cured breast prosthesis.

### BRIEF DESCRIPTION OF THE FIGURES OF THE DRAWINGS

FIG. 1 is a side schematic view of a first embodiment of a combination breast prosthesis.

FIG. 2 is a side schematic view of a second embodiment of a combination breast prosthesis.

FIGS. 3A-3E are a series of schematic diagrams showing one method of making a breast prosthesis.

FIGS. 4A-4C are schematic diagrams showing a second method of making a breast prosthesis.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

The invention includes an external breast prosthesis made of both encapsulated silicone gel and silicone elastomer, combining the benefits of both technologies, such as the lifelike feel of silicone gel and the natural tackiness of silicone elastomer for improved comfort while wearing.

Applicant has discovered that the two above-described breast prosthesis technologies can be combined in a straightforward production process to create a breast prosthesis made of an encapsulated silicone gel and silicone elastomer with good adhesion between all components and expected good durability.

In one representative embodiment, the front of the breast prosthesis is made of encapsulated silicone gel to mimic the feel and movement of fatty tissue. The back of the breast prosthesis is made of silicone elastomer, so that the wearer can securely wear the prosthesis against the chest wall without the requirement of a pocketed bra. The silicone elastomer is not an adhesive gel, so it does not have the same limitations of an attachable gel breast prosthesis in regards to health insurance coverage and maintenance. The wearer can also place the prosthesis into a bra pocket, if desired, without having to use a cover. The firm elastomer back can also be molded into textured shapes that can help with ventilation for perspiration or have a massaging effect. The firm elastomer can maintain a molded shape more so than molded, soft gel.

In another representative embodiment, the front of the breast prosthesis is made of silicone elastomer and the back made of silicone gel. This gives the prosthesis a smooth, wrinkle free look on the front, which is desired by some breast prosthesis wearers. The firm elastomer front maintains the same projection, regardless of the wearer's bodily orientation. This could be desired when the breast prosthesis needs to match the same shape and non-moving behavior of a reconstructed breast with firm implants. The firm elastomer front is coupled with a soft gel back for improved comfort. The soft gel can also have a reduced density. Additionally, the silicone gel does not need to be “foamed” so there is no chance of moisture being trapped within the prosthesis.

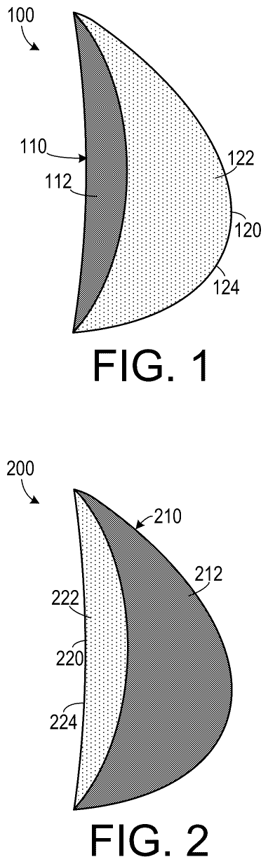

As shown in FIG. 1, one representative embodiment of a breast prosthesis 100 includes a front part 120 that is made of a silicone gel 122 that is encapsulated in a polymer film 124. Attached to the front part 120 is a back part 110 that is made from a silicone elastomer 112.

A second representative embodiment, as shown in FIG. 2, is a breast prosthesis 200 that includes a front part 210 that is made from a silicone elastomer 212. Affixed to the front part 210 is a back part 220 that is made of a silicone gel 222 that is encapsulated in a polymer film 224.

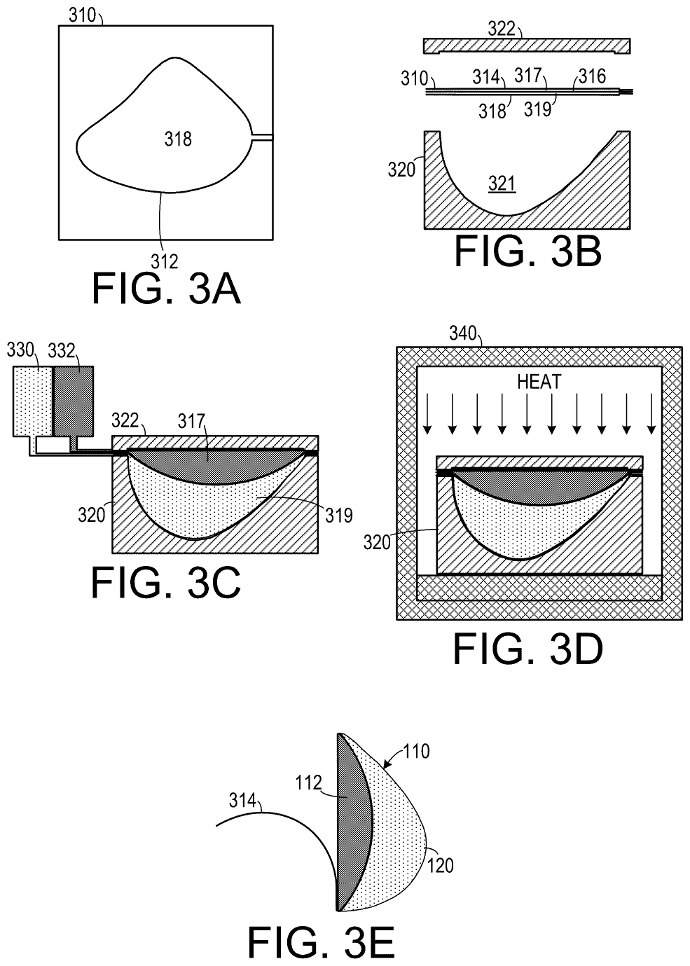

As shown in FIGS. 3A-3E, in one method of making a breast prosthesis 110 with a gel back part 112 and an elastomer front part 120, a two chamber bag 310 is made with a back layer of polytetrafluoroethylene (“PTFE”, such as Teflon®) 314, a middle layer of polyurethane 316 and a front layer of polyurethane 318. A silicone cross-linker (such as methyl hydrogen polysiloxane that is end-capped with trimethyl siloxy groups) can be sprayed onto the polyurethane sheets to improve adhesion of the gel and elastomer materials.

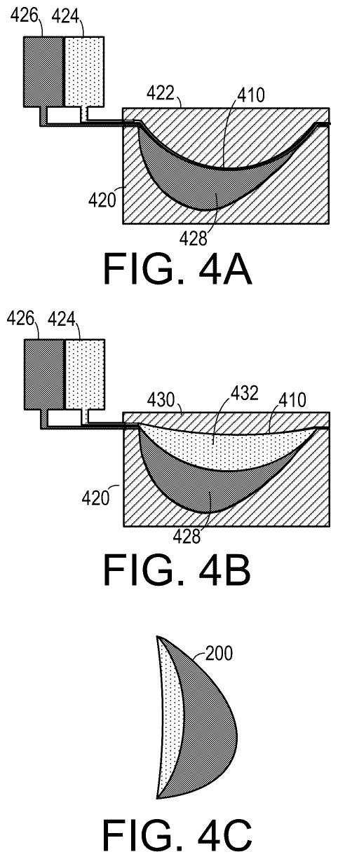

As shown in FIGS. 4A-4C, in a method of making a breast prosthesis 200 with a gel back part 220 and an elastomer front part 212, a two chamber polyurethane bag 410 is placed in a mold 420 and a back 422 with a deep shell shape is placed on the bag. Elastomer material 426 is injected into the front chamber 428. The elastomer material is cured (which can be accomplished at room temperature by the addition of a platinum-type catalyst). The back 422 with a deep shell shape is removed and a less projected or flat back 430 is placed over the bag 410 and silicone gel material 424 is injected into the back chamber 432 defined by the bag 410. The silicone gel material 424 is cured in an oven. This also heat seals the bag injection inlet shut. Once cured, the resulting breast form 200 is removed from the mold 420.

The front and middle layers are heat sealed with a breast form footprint 312 so as to form a front chamber 319 therebetween. The bag 310 is placed in mold 320 that defines a breast form-shaped cavity 321 and a top is placed against the bag to hold it in place and to seal the PTFE layer 314 against the middle layer 316 so as to form a back chamber 317. The silicone gel material 330 is injected into the front chamber 319 and the elastomer material 332 is injected into the back chamber 319. The mold 320 is placed into an oven 340 and the materials are cured. Also, curing can be a multi-step process in which the gel material 330 is injected first and then cured, and then the elastomer material 322 is injected and then cured. Alternately, the materials can be chemically cured without the use of an oven by adding a curing substance to the silicone elastomer material and the silicone gel material before injecting them into the chambers.

Once the materials have cured, the breast form is removed from the mold and the PTFE layer 314 is peeled away exposing the elastomer 112 and discarded. The breast prosthesis 110 is now ready for use. A similar process can be employed for making a prosthesis of the type shown in FIG. 2 by placing the PTFE layer in the front and injecting the elastomer in the front chamber formed between the PTFE layer and the middle layer, then injecting the gel into the back chamber.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112(f) unless the words “means for” or “step for” are explicitly used in the particular claim. The above-described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A breast prosthesis, comprising:
(a) front part that is made of a silicone gel that is encapsulated in a polymer film; and
(b) a back part, affixed thereto, that is made from a silicone elastomer.

2. A breast prosthesis, comprising:
(a) a front part that is made from a silicone elastomer; and
(b) a back part, affixed thereto, that is made of a silicone gel that is encapsulated in a polymer film.

3. A method of making a breast prosthesis, comprising the steps of:
(a) making a two chamber bag that includes a back layer of polytetrafluoroethylene (“PTFE”), a middle layer of polyurethane and a front layer of polyurethane;
(b) heat sealing the front layer to the middle layer along a breast form footprint so as to define a front chamber therebetween;
(c) placing the bag into a mold that defines a breast form-shaped cavity and placing a top against the mold to hold the PTFE layer in place so as to define a back chamber between the middle layer and the PTFE layer;
(d) injecting a silicone gel material into the front chamber and injecting a silicone elastomer material into the back chamber;
(e) curing the silicone gel material and the silicone elastomer material so as to form a cured breast prosthesis;
(f) removing the cured breast form from the mold and removing the PTFE layer from cured breast prosthesis.

4. The method of claim 3, further comprising the step of spraying a silicone cross-linker onto the front layer to the middle layer prior to the injecting step so as to promote adhesion of the silicone gel material and the silicone elastomer material to polyurethane.

5. The method of claim 4, wherein the silicone cross-linker comprises methyl hydrogen polysiloxane that is end-capped with trimethyl siloxy groups.

6. The method of claim 3, wherein the curing step comprises placing the mold into an oven and heating the oven.

7. The method of claim 3, wherein the curing step comprises adding a curing substance to the silicone elastomer material and the silicone gel material prior to the injecting step.

8. A method of making a breast prosthesis, comprising the steps of:
(a) making a two chamber bag that includes three layers polyurethane;
(b) heat sealing the polyurethane layers along a breast form footprint so as to define a back chamber therebetween;
(c) injecting a silicone elastomer material into the front chamber and allowing the silicone elastomer material to cure;
(d) injecting a silicone gel material into the back chamber;
(e) curing the silicone gel material so as to form a cured breast prosthesis;
(f) removing the cured breast form from the mold.

9. The method of claim 8, further comprising the step of spraying a silicone cross-linker onto the back layer to the middle layer prior to the injecting step so as to promote adhesion of the silicone gel material and the silicone elastomer material to polyurethane.

10. The method of claim 9, wherein the silicone cross-linker comprises methyl hydrogen polysiloxane that is end-capped with trimethyl siloxy groups.

11. The method of claim 8, wherein the step of curing the silicone elastomer material comprises adding a catalyst to the silicone elastomer material.

12. The method of claim 11, wherein the catalyst comprises a platinum-type catalyst.

13. The method of claim 8, wherein the step of curing the silicone gel material comprises placing the mold into an oven and heating the oven.

14. The method of claim 8, wherein the curing step comprises adding a curing substance to the silicone elastomer material and the silicone gel material prior to the injecting step.
